(19) United States
(12) Patent Application Publication
(10) Pub. No.: US 2004/0102438 A1
(43) Pub. Date: May 27, 2004

(54) Gaba-receptor modulators for the treatment of neurodegenerative diseases of the eye

(76) Inventors: Christopher Brückner, (US); Christoph Kessler, Berlin (DE); Cornelia Remia-Hevia, Falkensee (DE); Helmut Allmeier, Falkensee (DE)

Correspondence Address: Law Department, Bausch & Lomb Incorporated, One Bausch & Lomb Place, Rochester, NY, 14604-2701, US

(21) Appl. No.: 10/467,897

(22) PCT Filed: Feb. 13, 2002
(86) PCT No.: PCT/EP02/01537

(30) Foreign Application Priority Data
Feb. 13, 2001 (DE) 101 06 470.5

(51) Int. Cl. A61K 31/5513; A61K 31/515; A61K 31/4745

(52) U.S. Cl. 514/221; 514/270; 514/291

(57) ABSTRACT

The invention relates to the use of pharmaceutical compositions comprising GABA-receptor modulators in ophthalmology, for the prevention and/or treatment of neurodegenerative diseases of the eye, whereby the GABA-receptor modulators are selected from the group comprising benzodiazepines, benzodiazepine-receptor ligands of various structures, beta-carbolines, barbiturates, barbituric acid derivatives and/or neurosteriods.

The present invention relates to the use of a pharmaceutical composition for the prevention and/or treatment of neurodegenerative diseases of the eye, together with suitable pharmaceutical compositions for this.

It is known that enhanced glutamate levels in the eye lead to serious damage of the nervous tissue of the eye. A result of the increased glutamate level in the eye can be a reduction in visual capacity including blindness. An increase in the intraocular glutamate level can be triggered e.g. by oxygen deficiency and/or an intraocular pressure increase.

Thus increased intraocular glutamate levels are mainly responsible for a number of eye diseases such as age-related macular degeneration, diabetic retinopathy, glaucoma and the ocular ischemia syndrome.

The pathologically most important glutamate receptor is the. NMDA receptor. The NMDA receptor acts voltage- and ligand-dependently simultaneously. At the normal resting potential of the neurone of −60 mV the ion channel of the receptor is sealed by Mg²⁺ ions, and a ligand cannot activate the receptor. On the depolarisation of the membrane, Mg²⁺ leaves the ion channel and the receptor can then be activated by a ligand, which leads to the inflow of Ca²⁺ and Na⁺ and an outflow of K⁺.

With intracellular cascade triggering, the Ca²⁺ assumes a central role, it having in high concentrations a toxic effect on cells. The term “excitotoxic effect” is conventionally also used in this context. An excessive activation of the NMDA receptor—the glutamate receptor is involved here, which is responsible for the bulk of the neurodegenerative diseases—produces an increased Ca²⁺ inflow, which, as already stated above, has a toxic effect on cells. Due to said Ca²⁺ inflow, apotosis and later necrosis processes are triggered, which finally lead to cell death.

In the advanced stage of a neurodegenerative disease the cells occupied by NMDA receptors mainly go dead. Said cells are then no longer accessible for a treatment with NMDA receptor antagonists, which leads with progressive neurodegeneration to NMDA receptor anatagonists losing more and more of their effectiveness.

Although the administration of NMDA receptor antagonists would be desirable for neuro-protective purposes, it has been shown in clinical tests that serious side-effects occur in this case, which have made a clinical introduction impossible. In addition, almost all NMDA receptor antagonists trigger moderately severe to severe psychiatric side-effects.

There is therefore a great need for a pharmaceutical composition that is usable for the neuro-protective treatment of the eye and prevents the aforementioned drawbacks of the prior art. In particular there is a need for a pharmaceutical composition suitable for treatment in neuro-protection which results in a clear reduction or prevention of the harming and devitalizing of neuronal cells.

It has now been found that the use of GABA-receptor-modulator-containing pharmaceutical compositions overcomes the described drawbacks of the prior art.

It is known in the prior art to use GABA agonists for reducing pressure in the eye. GABA agonists have the great disadvantage, however, that they also act in the absence of a neurotransmitter. Agonists therefore possess a high toxic potential, since an increased dosage of agonists can lead linearly to a reinforcement of inhibition up to anesthesia and respiratory arrest. An increase in the effect of modulators is limited by the amount of transmitter present and cannot be increased arbitrarily.

The object is achieved according to the invention by the use of GABA-receptor-modulators in ophthalmology for the prevention and/or treatment of neurodegenerative diseases of the eye, the GABA-receptor-modulators being selected from the group including benzodiazepines, benzodiazepine-receptor ligands of varying structure, beta-carbolines, barbiturates; barbituric acid derivatives and/or neurosteroids. The object of the present invention is therefore to achieve a glutamate protection by modulation of the opposing transmitter system, namely of the GABA-ergic system.

There are regarded as neuro-protective therapy for the purpose of this invention those forms of treatment which lead to a reduction or prevention of the harming and devitalizing of neuronal cells.

“GABA” stands for gamma-amino-butyric acid. GABA arises within the glutamate metabolism and represents the most important inhibitory transmitter in the nervous system of the mammal. A lack of GABA or a suppression of the GABA-ergic system leads in most cases to spasms and epileptic convulsions up to cell death.

GABA is the physiological antagonist of glutamate. An activation of the GABA-receptor produces an inflow of Cl⁻-ions, which changes the membrane potential in negative direction, and reduces the probability of an action potential. GABA is therefore described as an inhibitory transmitter.

Without being tied to a particular theory, it is assumed that a reinforcement of the GABA-ergic system leads to a hyperpolarisation of the cell. This makes a depolarisation or the spread of an action potential more difficult. The NMDA receptor becomes permeable for Ca²⁺ only if the cell membrane is depolarised. In other words, an application of GABA-reinforcing agents leads in this way to an inhibition of the NMDA receptor. By the hyperpolarisation of the cell membrane, glutamate receptor-independent, voltage-dependent Ca²⁺ channels are prevented from being activated. Said channels are not covered by an NMDA-receptor antagonist. Said two effects are inter alia responsible for GABA-receptor-modulators being usable according to the invention for the use of neurodegenerative diseases of the eye.

Thus, GABA-receptor-modulators modify the affinity and effectiveness of GABA at the receptor. In the absence of GABA, GABA-receptor-modulators are ineffective. The administration of GABA-receptor-modulators is therefore harmless compared with the administration of agonists, since agonists have an increasingly stronger effect with a rise in administered concentration independently of the amount of neurotransmitter present, and thus cause cell damage up to cell death. The administration of GABA-receptor-modulators therefore possesses a far smaller toxic risk compared with GABA agonists.

GABA-receptor-modulators for the purpose of this invention are substances that modulate the affinity/effectiveness of GABA present at the synaptic GABA-receptor. The GABA-receptor-modulators used according to the invention are so-called positive modulators, i.e. they reinforce the synaptic affinity/effectiveness of GABA.

Pharmaceutically acceptable salts of the claimed GABA-receptor-modulators include in particular salts of benzodiazepines, beta-carbolines, barbiturates, barbiturate acid derivatives and/or neurosteroids.

Particularly suitable according to the invention is the use of benzodiazepine derivatives selected from the group comprising aiprazolam, bentazepam, bromazepam, brotizolam, cannazepam, chiordiazepoxide, clobazam, clonazepam, cinolazepam, clotiazepam, cloxazolam, clozapin, delorazepam, diazepam, dibenzepin, dipotassium chlorazepat, estazolam, ethyl-loflazepat, etizolam, fludiazepam, flumazenil, flunitrazepam, flurazepamI 1HCl, flutoprazepam, hal azeparn, haloxazol am, ketazolam, lorazepam, loprazolam, lormetazepam, medazepam, metaclazepam, mexozolam, midazolam-HCl, nimetazepam, nitrazepam, nordazepam, oxazepam-tazepam, oxazolam, pinazepam, prazepam, quazepam, temazepam, tetrazepam, tofisopam, triazolam and zolezepam as well as pharmaceutically compatible salts.

Particularly suitable according to the invention is the use of benzodiazepine-receptor-ligands of varying chemical structure, selected from the group comprising RWJ46771 (a pyrido-[1,2-a]-benzimidazole), SX-3228 (a 1,6-naphthydrin-2(1H)one derivative, Y-23684 (a pyridazinone), bretazenil, imidazenil, nabanezil, sarmazenil, zaleplon, zolpidem and zopielon.

Particularly suitable beta-carbolines usable according to the invention comprise abecarnil, 3,4-dihydro-beta-carboline, gedocarnil, 1-methyl-1-vinyl-2,3,4-trihydro-beta-carboline-3-carboxylic acid, 6-methoxy-1,2,3,4-tetrahydro-beta-carboline, N-BOC-L-1,2,3,4-tetrahydro-beta-carboline-3-carboxylic acid, tryptoline, pinoline, methoxyharmalan, tetrahydro-beta-carboline (THBC), 1-methyl-THBC, 6-methoxy-THBC, 6-hydroxy-THBC, 6-methoxyharmalan, norharman and/or 3,4-dihydro-beta-carboline as well as pharmaceutically compatible salts thereof.

According to the invention barbiturates or barbituric acid derivatives particularly suitable for the use are selected from the group comprising barbexaclon, dipropyl barbituric acid, eunarcon, hexobarbital, mephobarbital, methohexital, Na-methohexital, pentobarbitall phenobarbital, primidone, 2,4,6(1H,3H,5)-pyrimidintrion, secbutabarbital and/or thiopental as well as pharmaceutically compatible salts thereof.

According to the invention neurosteriods particularly suitable for the use are selected from the group comprising allopregnenolone, 3beta-hydroxyandrost-5-en-17-on-3-sulfate, dehydroepianrosterone, eltanolone, ethinylestradiol, 5-pregnen-3beta-ol-20 on-sulfate, pregnenolone and/or progesterone as well as pharmaceutically salts thereof.

The most suitable GABA-receptor-modulators usable according to the invention are flunitrazepam, midazolam, phenobarbital, abecarnil and/or dehydroepiandrosterone as well as pharmaceutically compatible salts thereof.

By pharmaceutically compatible salts in the context of this invention are understood all salts known in the prior art whose pharmacological effectiveness does not affect the effectiveness of the active ingredients disadvantageously, i.e. impair it in a manner endangering health.

There can be used in particular according to the invention pharmaceutically compatible salts selected from the group comprising organic and/or inorganic acids and bases derived therefrom. Salts usable according to the invention can be a mono-, di- and/or polyvalent ion. Particularly preferred are alkaline and alkaline earth salts such as Na⁺, Ca²⁺, K⁺ and/or Mg²⁺. Salts of organic amines such as mono-, di- and triamines and ethanolamines can also be used. Salts can also be formed with caffein, tromethamine and/or similar. In cases where a nitro group is required to form a salt, the latter can be formed with any arbitrary organic and/or inorganic substance such as methyl iodite. Still more preferable are salts that are formed with inorganic acids such as chloric acid, sulfuric acid and/or phosphoric acid. 1-, 2- and 3-valent acids can thereby be formed.

In addition, all physically/chemically possible isomers of the GABA-receptor-modulators usable according to the invention are claimed.

The GABA-receptor-modulators can be used according to the invention in ophthalmology for the prevention and/or treatment of neurodegenerative diseases.

The use of GABA-receptor-modulators for diseases of the retina is particularly preferred.

The use of GABA-receptor-modulators in retinopathy of prematurity such as retinopathia praematurorum and/or in retrolental fibroplasia is suitable according to the invention.

The GABA-receptor-modulators are usable according to the invention for the treatment of vascular diseases of the retina such as retinopathia angiospastica, arteriosclerotic retinopathy, eclamptic retinopathy, diseases caused by occlusions of the aorta carotis, periphlebitis retinae, diabetic retinopathy, non-proliferative diabetic retinopathy, proliferative diabetic retinopathy, diabetic maculopathy, carcinoma-associated retinopathy and/or retinopathy due to radiation trauma.

The GABA-receptor-modulators can also be used according to the invention in diseases caused by venous and/or arterial vascular occlusions, such as diseases caused by branch vein, occlusions, central vein occlusion, arterial occlusion, amaurosis fugax, occlusion of venule of retina, chronic ocular ischemia, sickle cell retinopathy, ocular ischernic syndrome and/or retinitis exsudativa.

The GABA-receptor-modulators can also be used for the prevention and/or treatment of macular degenerations, such as moist and dry macular degeneration, acquired macu lar degenerations, age-related macular degeneration, retinopathia centralis serosa, rmyopic macular changes, cystiform macular edema, vasiform stripes, toxic macular diseases, maculaforamen, exudative maculopathies due to other causes, chlorioretinopathy centralis serosa, cystiform macular edema, submacular bleeding, hereditary macular and retinal degenerations, juvenile macular degenerations, vitelline macular degenerations, albinism, storage diseases, amaurotic idiocy, sphingolipidoses, Tay-Sachs disease, Niemann-Pick disease, gangliosidosis, Gaucher's disease, Spielmeyer-Vogt-Stock disease and/or in Sandhoffs disease.

The GABA-receptor-modulators can also be used according to the invention for the prevention and/or treatment of traumatic retinal changes such as contusion of the eye, perforating eye injuries, siderosis/hemidosis, chalcosis, bums, retinopathia traumatica and/or injury to the retina from light.

In addition, the GABA-receptor-modulators can also be used according to the invention for retinoschisis.

The use of GABA-receptor-modulators is also suitable according to the invention for the prevention and/or treatment of diseases of the choroid, such as hyalin deposits and/or choroideremia.

The GABA-receptor-modulators can also be used according to the invention for diseases of the optic nerve. The latter include trauma to the nervus opticus caused by intoxications such as tobacco-alcohol trauma, trauma caused by methyl alcohol, trauma caused by ethambutol, trauma caused by quinine, arsenic, lead and/or bromine.

The GABA-receptor-modulators can also be used according to the invention for anterior ischemic optic neuropathy, such as apoplexia papillae and/or Horton's syndrome.

The GABA-receptor-modulators can also be used for the prevention and/or treatment of an optic atrophy, such as traumatic optic atrophy, optic atrophy caused by tumour pressure, hereditary optic atrophy, liver optic atrophy, secondary optic atrophy, optic atrophy after papillitis/retrobulbar neuritis, optic atrophy of uncertain origin, glaucomatous optic atrophy and/or changes to the optic nerve head.

In a further preferred embodiment GABA-receptor-modulators are used according to the invention for the treatment of glaucoma, such as primary glaucoma, Donders' glaucoma, primary Donders' glaucoma, normotension glaucoma, angle-closure glaucoma, acute angle-closure glaucoma, intermittent angle-closure glaucoma, subacute angle-closure glaucoma, chronic angle-closure glaucoma, plateau iris and/or nanopthhalmos.

The GABA-receptor-modulators are also usable according to the invention for congenital glaucoma and premature glaucoma, such as cornea-angle of chamber-iris dysgeneses, Lowe's syndrome, Sturge-Weber syndrome, neurofibromatosis, Rubinstein-Taybi syndrome, Pierre Rubin syndrome, Ota's nevus, trisomy, Marfan syndrome, Turner's syndrome, aniridia, homocystinuria, intraocular tumours, orbital lymphangioma, retinopathia praematurorum, persistent hyperplastic primary vitreous body, ectopia lensis, intraocular inflammation, cortisone therapy, myopia with pigmentary glaucoma, rubella embryopathy, cataract extraction and/or for treatment of blunt or acute trauma.

The use of GABA-receptor-modulators is also suitable according to the invention for the treatment of glaucoma simplex, such as glaucoma with aphakia and pseudoaphakia, glaucoma with diabetes mellitus; glaucoma and dystrophia endotheliasis, hypersecretion glaucoma, glaucoma in pregnancy, higher myopia and/or juvenile glaucoma.

GABA-receptor-modulators can also be used according to the invention for the treatment of secondary glaucoma, such as traumatic and postoperative glaucoma, secondary Donders' glaucoma, secondary angle-closure glaucoma, steroid-induced glaucoma, glaucoma after inflammation, phacolytic glaucoma, Posner-Schlossman syndrome, heterochromic cyclitis, ghost cell glaucoma, hemolytic glaucoma, neurofibromatosis, siderosis, glaucoma caused by regeneration of vessels, glaucoma caused by cortisone administration, pigmentary glaucoma, pseudoexfoliation glaucoma, glaucoma with anterior uveitis, glaucoma with Fuchs heterochromia, Grant's syndrome, glaucoma after contusions, chamber angle abnormalities of non-traumatic origin, erythroclastic glaucoma, silcione glaucoma, lens-related glaucoma, phacotopical glaucoma, phacomorphic glaucoma, glaucoma caused by free lens material, pseudoexfoliation glaucoma, phacogenic uveitis, glaucoma with anterior uveitis, malignant glaucoma and/or for glaucoma caused by increased episcleral venous pressure.

It was also found according to the invention that the GABA-receptor-modulators are suitable for the treatment of ocular hypertension, for example for the primary and secondary form.

According to the invention the pharmaceutical compositions containing GABA-receptor-modulators can be administered in liquid, gel and/or solid form. Preferably the pharmaceutical composition containing GABA-receptor-modulators, if it is in free-flowing form, is to be administered as drops, topically or systemically in solid form. Preferably the GABA-receptor-modulators are provided in pharmaceutical compositions suitable for use in a concentration of 0.0001 to 5 wt %, referred to the total composition.

Topical applications are preferred to systemic applications according to the invention, since with topical application a substantially higher concentration of active ingredient takes effect directly on the eye.

The ophthalmological compositions containing GABA-receptor-modulators usable according to the invention can contain preservatives, means for adjusting the tonicity, buffers for adjusting the pH value, antioxidants, viscosity-regulating substances and/or further conventionally usable auxiliary substances.

Preferred preservatives are selected from the group comprising benzalkonium chloride, chlorobutanol, thiomersal, phenylmercuric acetate, cetrimide, EDTA and/or phenylmercuric nitrate.

Suitable support materials according to the invention are selected from the group comprising polyvinyl alcohol, polyvinylpyrrolidone, hydroxypropyl methylcellulose, Poloxamer, carboxymethylcellulose, hydroxyethylcellulose, cyclodextrins and/or water.

Means for adjusting the tonicity are selected from the group comprising salts, preferably sodium chloride and/or potassium chloride, mannitol, glycerol and similar.

Acetate, citrate, phosphate and borate buffers can be used for the pH value adjustment. Acids and/or bases can naturally also be used if necessary.

Ophthalmologically acceptable antioxidants are selected from the group comprising sodium metabisulfite, sodium thiosulfite, acetyl cysteine, butylated hydroxyanisoles and/or butylated hydroxytoluenes.

Carbomers in particular can be used as viscosity-regulating substances.

In the case of the use according to the invention of the pharmaceutical compositions containing GABA-receptor-modulators, the latter are applied topically and/or systemically for the neuroprotective prophylaxis and/or treatment of mammals' eyes, particularly in the case of humans. In the case of topical application, said pharmaceutical compositions containing GABA-receptor-modulators are used in an ophthalmologically compatible preparation, e.g. as eye drops, eye gel or ophthalmic ointment. A suitable pharmaceutical composition is particularly suitable if it exerts a harmful or dangerous influence on the eye neither in the acute nor in the chronic application. Particularly preferred are pharmaceutical compositions that cause no eye irritation such inflammation, itching or similar.

It is particularly preferred to apply a pharmaceutical composition containing GABA-receptor-modulators to the eye in the form of a solution or a gel.

In cell cultures and in freshly prepared retina cells of rats and rabbits, it was shown experimentally that the use of GABA-receptor-modulators led to a significant drop in the number of devitalized cells under excitotoxic stress.

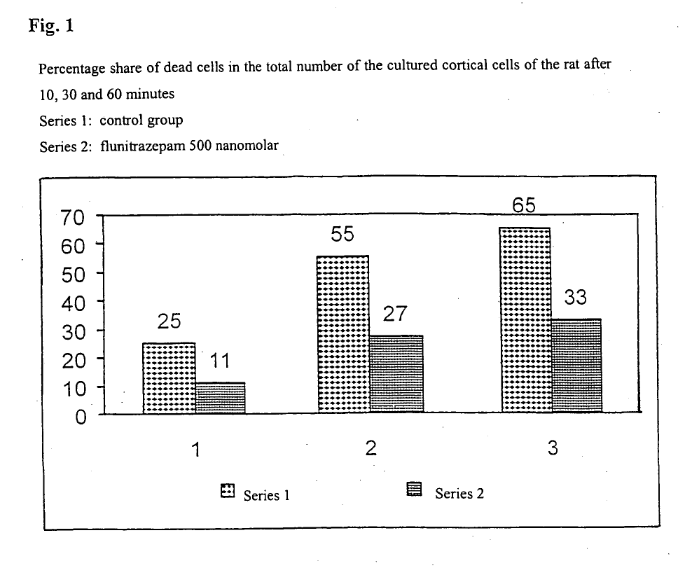

FIG. 1 shows after the administration of flunitrazepam the reduction in the number of dying cells in cell cultures of cultured cortical cells of the rat after 10, 30 and 60 minutes compared with the control group.

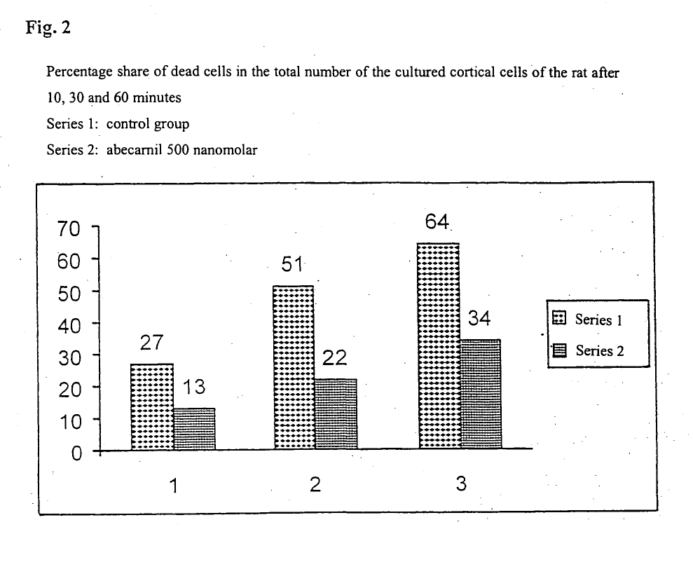

FIG. 2 shows after the administration of abecamil the reduction in the number of dying cells in cell cultures of cultured cortical cells of the rat after 10, 30 and 60 minutes compared with the control group.

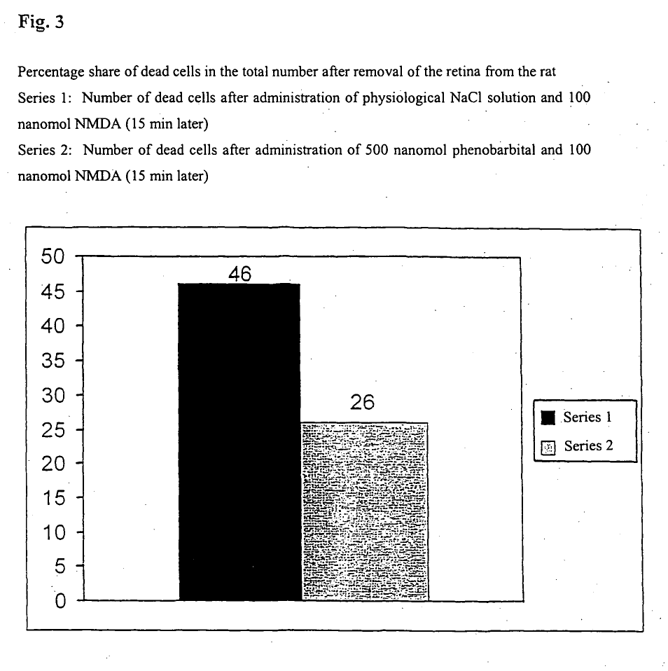

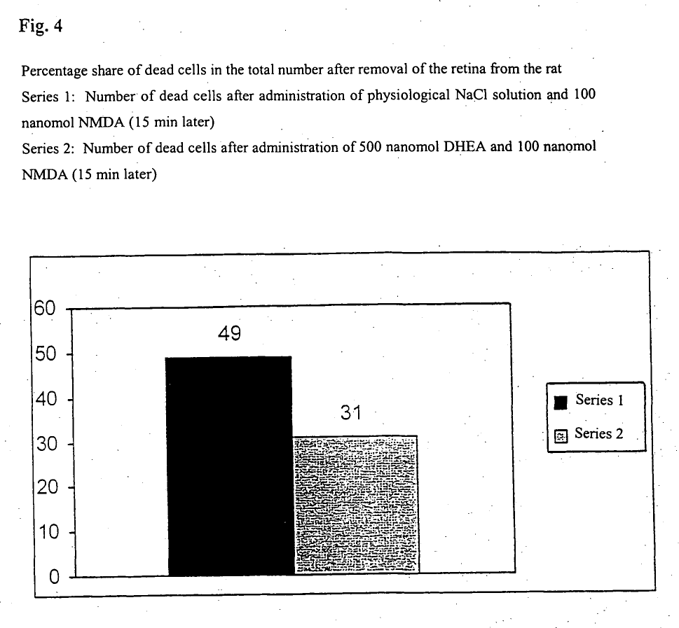

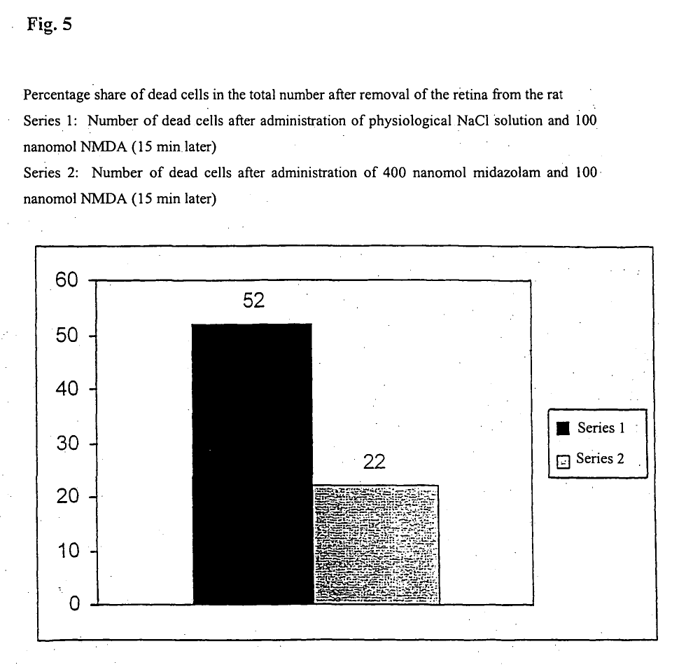

FIGS. 3 to 5 show after the administration of phenobarbital (FIG. 3), dehydroepiandrosterone (DHEA; FIG. 4) and midazolam (FIG. 5) the number of dead cells in retina preparations of the rat, after injection of a 100 nanomolar NMDA solution into the eyeball of an anesthetised rat.

Pharmaceutical compositions suitable for the use according to the invention possess 0.0001-5 wt %, for preference 0.001-3 wt %, more preferably 0.01-2 wt %, more preferably still 0.05-1.5 wt % and most preferably of all 0.1-1 wt % of active ingredient, referred to the total pharmaceutical composition.

The proportion of preservative comes to 0-0.5 wt %, for preference >0-0.1 wt %, preferably 0.001-0.3 wt % and more preferably still 0:01-0.05 wt %, referred to the total pharmaceutical composition.

The proportion of support substance comes to 0-99 wt %, for preference >0-80 wt %, preferably 1-50 wt %, more preferably still 5-40 wt % and most preferably of all 10-30 wt %, referred to the total pharmaceutical composition.

The proportion of the substances for adjusting the isotonicity comes to 1-10 wt %, preferably 2-8 wt % and particularly preferably 3-5 wt %, referred to the total pharmaceutical composition.

The proportion of buffer substances comes to 0.01-10 wt %, for preference 0.05-5 wt %, preferably 0.01-3 wt % and more preferably still 0.1-2 wt %, referred to the total pharmaceutical composition.

The pH value of the pharmaceutical composition containing GABA-receptor-modulators lies preferably in the range between pH 4.5 and 7.5, for preference between pH 5 and 7.3, preferably between pH 6 and 7.1 and more preferably between 6.5 and 7.0.

The proportion of antioxidants comes preferably to 0-10 wt %, for preference >0-8 wt %, preferably 0.005-5 wt %, more preferably still 0.05-2 wt % and more preferably 0.1-1 wt %, referred to the total pharmaceutical composition.

Suitable pharmaceutical compositions containing GABA-receptor-modulators are listed in the following examples:

Properties of the Active Ingredients Used According to the Invention:

Benzodiazepines are lipophilic

Midazolam is water-soluble

Beta-carbolines are lipophilic

Phenobarbital-Na and pentobarbital-Na are hydrophilic

Steriods are lipophilic

### EXAMPLE 1

| Eye drop (general formula): | |
|---|---|
| Pharmaceutical | 0.0001-5% |
| Preservative | 0-0.1% |
| Support substance | 0-40% |
| Substance for adjusting the isotonicity | 1-10% |
| Buffer | 0.01-10% |
| Substance for adjusting the pH value | q.s. |
| from 4.5 to 7.5 | |
| Antioxidant | q.s. |

### EXAMPLE 2

| Oily diazepam eye drop | |
|---|---|
| Diazepam | 0.5 |
| Hydrarg. bichlorate | 0.04 |
| EtOH | 1.0 |
| Oleum ricini | 98.46 |

### EXAMPLE 3

| Oily diazepam eye drop | |
|---|---|
| Diazepam | 1.0 |
| Palmitoyl ascorbic acid | 0.01 |
| Oleum ricini steril. | ad 100 ml |

### EXAMPLE 4

| Aqueous eye drop: | |
|---|---|
| 1 ml of solution contains | |
| Substance e.g. midazolam | 3.42 |
| BAC | 0.1 |
| Na-dihydrogenphosphate dihydrate | 8.15 |
| Di-Na-phosphate-dodecahydrate | 29.21 |
| Water f inj | ad 1000 |

### EXAMPLE 5

| Ophthalmic ointment | |
|---|---|
| Diazepam | 2.0 |
| Thiomersal. sol. 0.002% | 15.0 |
| Oculent. Simplex | ad 100 |
| [Oculent. Simplex: | |
| Cholesterolum | 1.0 |
| Paraffin. subliquid. | 42.5 |
| Vaselin. Album | ad 100.0] |

### EXAMPLE 6

| Eye gel: | |
|---|---|
| Phenobarbital-Na | 0.001 |
| Hydroxyethylcellulose | 20 |
| BAC | 0.1 |
| Na-EDTA | 1 |
| NaCl | 9 |
| NaOH (1 molar solution) | q.s. |
| Aqua ad inject. | ad 1000 |

### EXAMPLE 7

| Parenteral lorazepam solution: | | |
|---|---|---|
| 1 mg | Lorazepam | 2 mg |
| | Benzyl alcohol | 20.9 mg |
| | Macrogol 400 | 202.5 mg |
| | Propylene glycol | ad 1000 mg |

### EXAMPLE 8

| Effervescent tablet: | |
|---|---|
| Midazolam | 5 mg |
| Sodium dihydrogen citrate, water-free | 1200 mg |
| Ascorbic acid | 240 mg |
| Citric acid, water free | 240 mg |
| Sodium hydrogen carbonate | 920 mg |
| Sodium carbonate, water-free | 200 mg |

### EXAMPLE 9

| Tablet: | |
|---|---|
| Abecarnil | 200 mg |
| Caffein | 50.00 mg |
| Maize starch | 340 mg |
| Microcrystalline cellulose | 60 mg |
| Stearin palmitic acid | 5 mg |
| Total: | 655.00 mg |

### Test 1

Dissociated cortical cells from 16-18 day old fetal rats were bred in 35 mm dishes. The nutrient medium contained during the first 7 days L-lutamine (4 mM), glucose (6 g/l), penicillin (100 U/ml), streptomycin (100 μg/ml) and 10% hormonally enriched medium, which [contained] transferrin (1 mg/ml), insulin (250 μg/ml), putrescine (600 μM), sodium selenite (0.3 μM), progesterone (0.2 μM) and estradio (0.1 μM). The dishes were stored in an atmosphere (5% CO₂ and 95% O₂) heated to 37 degrees Celsius. After 7 days the hormone-containing nutrient medium was siphoned off and replaced with another medium that was without the hormonal addition but in other respects possessed the same composition.

To form the control group (Series 1), half of the dishes were supplemented with the nutrient medium 10 nm L-glutamate and incubated under normotoxic conditions for 4 hours.

To determine the neuroprotective properties, the other half of the dishes were supplemented with the nutrient medium flunitrazepam 500 nm and 10 nm L-glutamate (Series 2) and likewise incubated under normotoxic conditions.

The number of surviving or devitalized cells was determined by determination of the amount of lactate-dehydrogenase (LDH) ejected into the cell medium. The LDH activity was determined photospectrometrically by measurement of the NADH metabolism at 340 nm.

### Test 2

Test 2 was carried out like Test 1, but instead of flunitrazepam the active ingredient abecamil 500 nanomolar was used.

In the diagrams of FIG. 1 and FIG. 2 the measured values were entered as follows: The measured value describes the number of dead cells after the test in relation to the total number of cells before the test in percent. Greater values designate a higher proportion of devitalized cells, 100% describes a complete cell loss.

### Test 3

To avoid systemic errors, the control measurements (Series 1) were carried out on the right eye for each animal, and the substance measurements (Series 2) on the left eye.

Series 1: In the control series (right eye) the same dosing scheme was applied as in Series 2, except that instead of the pharmaceutical solution containing the active ingredient phenobarbital 500 nanomol the same pharmaceutical solution without active ingredient was injected. In the case of the injection into the eyeball, 100 nM of NMDA in 5 μl of a sodium-phosphate buffer solution without pharmaceutical were injected into the control eye.

Series 2: One day before the NMDA injection 50% of the rats (female siblings) with a weight of 200-250 g received 5 μl drops of the pharmaceutical solution in two administrations, one in the early morning and one late afternoon, into the left eye. Each administration consisted of a drop of pharmaceutical solution with a second drop 30 minutes later. On the morning of the glutamate injection the rats received a 5 μl injection of the pharmaceutical solution, and were then anesthetised with 0.3 ml kg⁻¹ of Hyponorm (Janssen, Grove, U.K.). After 30 minutes 5 μl of the pharmaceutical solution were injected once again into each eye. 15 minutes later 100 nM of NMDA in 5 μl of a sodium-phosphate buffer solution were injected into the vitreous body. On the afternoon of the same day 2 drops of the pharmaceutical solution were administered into each eye. In the following 4 days 2 drops of the pharmaceutical solution were injected into the eye in the morning and evening respectively.

On the afternoon of the fourth day the animals were again anesthetised (0.3 ml kg⁻¹ of Hyponorm, Janssen, Grove, U.K.) and the retinas were removed for further immunohistochemical examination. To this end the retinas were fixed in 2% paraformaldehyde solution for 45 minutes and then frozen in 30% saccharose solution. Frozen sections (10 μm) were cut off at a distance of approx. 5 mm from the optic nerve. and observed on a gelatine-coated object slide. The sections were then further processed for localization of the Thy-1.

### Test 4

Test 4 was carried out like Test 3, except that instead of 500 nanomol phenobarbital the active ingredient dehydroepiandrosterone 500 nanomol (DHEA) was used.

### Test 5

Test 5 was carried out like Test 4, except that instead of dehydroepiandrosterone 500 nanomol (DHEA) the active ingredient Midazolam 500 nanomol was used.

In the diagrams of FIGS. 3, 4 and 5 the measured values were entered as follows: The measured value describes the number of dead cells after the test in relation to the total number of cells before the test in percent. Greater values designate a higher proportion of devitalized cells, 100% describes a complete cell loss.

1. Use of GABA-receptor-modulators to produce pharmaceutical compositions in ophthalmology for the prevention and/or treatment of neurodegenerative diseases of the eye, characterised in that the GABA-receptor-modulators are selected from the group comprising benzodiazepines, benzodiazepine-receptor ligands of varying structure, beta-carbolines, barbiturates, barbituric acid derivatives and/or neurosteroids.

2. Use of the pharmaceutical compositions containing GABA-receptor-modulators according to claim 1 for the treatment of neurodegenerative diseases, comprising retinopathy, vascular diseases of the retina, disease caused by venous and arterial vascular occlusions, macular degenerations, traumatic retinal changes, retinoschisis, diseases of the choroid, diseases of the optic nerve, anterior ischemic optic neuropathy, glaucoma, primary glaucoma, congenital glaucoma, premature glaucoma, glaucoma-simplex, secondary glaucoma and/or ocular hypertension.

3. Use of the pharmaceutical compositions containing GABA-receptor-modulators according to claim 1 or 2, in which the GABA-receptor-modulators include preferably flunitrazepam, midazolam-HCl, phenobarbital and/or dehydro-epiandrosterone as well as pharmaceutically acceptable salts thereof.

4. Use of the pharmaceutical composition according to any one of the preceding claims, in which the content of GABA-receptor-modulator comes to 0.0001 to 10 wt %, preferably 0.01 to 5 wt % and still more preferably 0.1 to 3 wt %, referred to the total composition.

5. Use of the pharmaceutical composition containing GABA-receptor-modulators according to any one of the preceding claims, in which the content of GABA-receptor-modulator [which] is present in a pharmaceutically active amount preferably comes to between 0.001 g and 1 g, more preferably still 0.01 to 0.8 and particularly preferably 0.05 to 0.6 g.

6. Use of the pharmaceutical composition containing GABA-receptor-modulators according to any one of the preceding claims for systemic and/or topical administration, in which the pharmaceutical composition is present in the form of a liquid, a gel and/or a solid.

7. Use of the pharmaceutical composition containing GABA-receptor-modulators according to any one of the preceding claims, in which the pharmaceutical composition comprises additives for adjusting the tonicity, buffers for the pH value adjustment, ophthalmologically acceptable antioxidants, gels, preservatives and/or substances for adjusting the viscosity.

8. Pharmaceutical composition comprising at least one GABA-receptor-modulator according to any one of the preceding claims suitable for the treatment of neurodegenerative diseases, in particular of the eye.
